(19) United States
(12) Patent Application Publication
(10) Pub. No.: US 2020/0262778 A1
(43) Pub. Date: Aug. 20, 2020

(54) PROCESS FOR PRODUCTION OF ALLYL ALCOHOL

(71) Applicant: SHELL OIL COMPANY, HOUSTON, TX (US)

(72) Inventors: Venkata Pramod CHODIMELLA, Enschede (NL); Jean Paul Andre Marie Joseph Ghislain LANGE, Amsterdam (NL); Kulathu Iyer SESHAN, Enschede (NL)

(21) Appl. No.: 16/335,497

(22) PCT Filed: Apr. 27, 2017
(86) PCT No.: PCT/EP2017/060108

§ 371 Date: Mar. 21, 2019

(30) Foreign Application Priority Data
Sep. 27, 2016 (EP) 16190861.1

(51) Int. Cl. C07C 51/16; C07C 29/60; B01J 23/04; B01J 21/06

(52) U.S. Cl. CPC C07C 51/16; B01J 21/066; B01J 23/04; C07C 29/60

(57) ABSTRACT

The invention relates to a process for producing allyl alcohol, the process comprising:
dehydrating a C3-oxygenate comprising monopropylene glycol or 1,3-propanediol;
wherein the dehydration is performed in the presence of a basic catalyst.

The C3-oxygenate may be derived from a biomass conversion process and subsequently converted to acrylic acid.

### FIELD OF THE INVENTION

The present invention relates to a process for the production of allyl alcohol and to a process for converting the allyl alcohol produced into acrylic acid.

### BACKGROUND OF THE INVENTION

Acrylic acid is a chemical for which the worldwide demand is high, about 5 Mt/a (million ton per annum) in 2008 and potentially rising to about 9 Mt/a by 2025. A known route for the production of acrylic acid comprises the oxidation of propene into acrolein (propenal) and then oxidation of the acrolein into acrylic acid. See for example “On the partial oxidation of propane and propene on mixed metal oxide catalysts” by M. M. Bettahar et al. in Applied Catalysis A: General, 145, 1996, p. 1-48. The overall reaction stoichiometry for this route is as follows:

CH₂=CHCH₃+1.5O₂→CH₂=CHCOOH+H₂O.

A disadvantage of the above-mentioned route for the production of acrylic acid is that two oxygen atoms have to be introduced into the propene by the use of an oxygen containing gas at high temperature (about 350° C.) and with release of a large amount of heat (about 600 kJ/mol). A further disadvantage is that propene has to be used which may be derived from propane. Both propene and propane are currently only readily available from fossil feedstocks and are therefore not renewable.

WO 2011/063363 discloses the conversion of malonate semialdehyde to 3-hydroxypropionic acid (3-HPA) and the subsequent conversion of the 3-HPA to acrylic acid.

WO 2001/16346 describes a process for producing 3-HPA from glycerol by fermentation. The 3-HPA may then be converted into acrylic acid.

EP 2495233 describes a process in which acrylic acid may be derived from biomass-derived lactic acid.

Cristina Della Pina et al., Green Chemistry, 2011, 13(7), 1624 discloses a synthesis of acrylic acid from 3-HPA and several routes to produce 3-HPA, with starting materials including 1,3-propanediol and glycerol.

Zhang, ACS Catalysis, 6(1), 143-150; 2016 discloses a method of oxidizing allyl alcohol to acrylic acid.

In addition to acrylic acid, monoethylene glycol is also a chemical for which the worldwide demand is high, about 20 Mt/a (million ton per annum) in 2008. Monoethylene glycol may be advantageously produced from sugar sources, such as sucrose, glucose, xylose or fructose and the corresponding polysaccharides, cellulose, hemicellulose, starch and inulin. A disadvantage of this route is that in addition to monoethylene glycol, a significant amount of monopropylene glycol is also formed. It may even be the case that two to three times more monopropylene glycol is formed than monoethylene glycol. See for example “Hydrogenolysis Goes Bio: From Carbohydrates and Sugar Alcohols to Platform Chemicals” by Agnieszka M. Ruppert et al. in Angew. Chem. Int. Ed., 2012, 51, p. 2564-2601.

In contrast to acrylic acid and monoethylene glycol, the worldwide demand for monopropylene glycol is not high, about 1.5 Mt/a (million ton per annum) in 2008 and projected to rise only to about 3 Mt/a by 2025. Currently, it is estimated that the worldwide demand for monoethylene glycol is ten times higher than that for monopropylene glycol. Because of this lower demand for monopropylene glycol, processes for converting sugar sources into monoethylene glycol may not be commercialized, unless the selectivity to monoethylene glycol would be drastically increased. Such selectivity increase is difficult to achieve. Consequently, there is currently a need in the art to valorize the monopropylene glycol that is automatically formed when transforming sugar sources into monoethylene glycol. A desired valorization could be an application wherein the monopropylene glycol is converted into a chemical for which the worldwide demand is high.

The above-mentioned monopropylene glycol is just one example of a C3-oxygenate. C3-oxygenates contain 3 carbon atoms and 1 or more oxygen atoms. There are C3-oxygenates other than monopropylene glycol, which may contain 1, 2 or 3 oxygen atoms and which may also be formed as undesired by-products in certain production processes such as biomass conversion processes. Such biomass conversion processes may include the aqueous phase reforming of sugars, as disclosed by N. Li et al. in Journal of Catalysis, 2010, 270, p. 48-59. Examples of such other C3-oxygenates include: 1-propanol, 2-propanol, propanal, acetone, monohydroxyacetone, 2-hydroxypropanal, dihydroxyacetone and 2,3-dihydroxypropanal.

Accordingly, it would be advantageous to valorize C3-oxygenates in general, including for example monopropylene glycol, which may be formed as undesired by-products in certain production processes such as biomass conversion processes.

WO 2014/108415 and WO 2014/108417 describe processes of converting monopropylene glycol to acrylic acid.

WO 2014/108418 describes a process of converting glycerin to acrylic acid. The latter two documents propose the combination of dehydration and oxidation routes, proceeding via the intermediates acrolein or propanal.

### SUMMARY OF THE INVENTION

Surprisingly, it is found that the above-mentioned C3-oxygenates can be valorized by means of a dehydration process to produce allyl alcohol which may then be converted into acrylic acid. Advantageously, in such way, the C3-oxygenate may be converted via allyl alcohol into acrylic acid, a chemical for which the worldwide demand is high. Further, advantageously, in such way, allyl alcohol and subsequently acrylic acid may be produced from a renewable feedstock since the starting C3-oxygenates may originate from biomass conversion processes. Further advantages of the present invention appear from the detailed description below.

Accordingly, the present invention relates to a process for producing allyl alcohol, the process comprising:

dehydrating a C3-oxygenate comprising 1,2- or 1,3-propanediol;

wherein the dehydration is performed in the presence of a basic catalyst.

The process of the invention may further comprise the step of converting the dehydrated C3-oxygenate comprising allyl alcohol into acrylic acid.

The present invention further relates to a process of producing acrylic acid, the process comprising:

dehydrating a C3-oxygenate comprising 1,2- or 1,3-propanediol in the presence of a basic catalyst to form allyl alcohol; and oxidizing the allyl alcohol to acrylic acid.

### BRIEF DESCRIPTION OF THE DRAWINGS

One or more embodiments of the invention will now be described, by way of example only, with reference to the accompanying drawings, in which:

FIG. 1 shows the yield of allyl alcohol from monopropylene glycol using different catalysts;

FIG. 2 shows the effect of KOH loading (%) of a catalyst on the conversion/yield (mol %) of allyl alcohol from monopropylene glycol;

FIG. 3 shows the effect of temperature on the conversion/yield (mol %) of allyl alcohol from monopropylene glycol;

FIG. 4 shows the effect of weight hourly space velocity (WHSV) on conversion and yield of various products from monopropylene glycol;

FIG. 5 shows the effect of dilution of the monopropylene glycol feed with water on the conversion/yield (mol %) of allyl alcohol from monopropylene glycol;

FIG. 6 shows the effect of temperature on allyl alcohol conversion when using a MoWVOₓ catalyst;

FIG. 7 shows the effect of contact time on allyl alcohol oxidation when using a MoWVOₓ catalyst at 340° C.;

FIG. 8 shows a general process scheme for the formation of crude acrylic acid from monopropylene glycol; and

FIG. 9 shows an advanced process flow scheme for the production of acrylic acid.

### DETAILED DESCRIPTION OF THE INVENTION

The present inventors have surprisingly found that use of a basic catalyst in the dehydration of a C3-oxygenate comprising 1,2- or 1,3-propane diol results in good yields of intermediates, especially allyl alcohol, which may then be converted into acrylic acid. While other intermediates may be formed in addition to allyl alcohol, such as propanal or 1-propanol, it is believed that in the presence of a basic catalyst, rapid isomerisation of allyl alcohol to propanal is suppressed.

The dehydrated C3-oxygenate, notably allyl alcohol, together with other intermediates such as propanal or 1-propanol, is preferably subsequently converted by oxidation into acrylic acid. Suppression of isomerisation is helpful in reducing by-products in the conversion.

The overall process, including the optional conversion to acrylic acid, is illustrated in the following general reaction scheme wherein the starting material for the last step of the process is a C3-allyl alcohol:

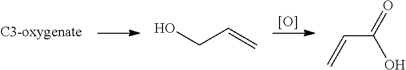

In the process of the invention, the starting material is a C3-oxygenate. Within the present specification, a C3-oxygenate means a compound which contains 3 carbon atoms and 2 oxygen atoms. The other atoms in such a C3-oxygenate are hydrogen atoms.

Thus the C3-oxygenates containing 2 oxygen atoms which may suitably be used in the present invention are monopropylene glycol (1,2-propanediol) and 1,3-propanediol.

Surprisingly, with the process of the present invention the above-mentioned disadvantages are avoided, while at the same time, advantageously, by means of the present invention C3-oxygenates, such as monopropylene glycol which may be formed as undesired by-products in certain production processes such as biomass conversion processes, may be valorized by transforming them into allyl alcohol which is then available for further conversion into a chemical for which the worldwide demand is indeed high, namely acrylic acid. Thus, in the process according to the invention, the C3-oxygenate preferably comprises a fraction obtained as a by-product in a biomass conversion process for production of monoethylene glycol. The biomass may typically comprise a sugar source.

The basic catalyst used in the process of the present invention preferably comprises an element with an electronegativity of less than 2.0, more preferably less than 1.5 and most preferably less than 1.0 (values based on Allred-Rochow scale).

Advantageously, the catalyst may comprise an element selected from Group 1 and/or Group 2 of the Periodic Table, with Na, K, Rb, Cs, Mg, Ca, Sr and Ba being preferred, especially K.

The basic catalyst may comprise a metal oxide or metal hydroxide. Preferably the metal oxide MOₓ, or the corresponding metal oxide where the catalyst is a metal hydroxide, has an electronegativity EN(MOₓ) of less than 2.5, less than 2.2, less than 2.0, less than 1.8, less than 1.6 or less than 1.4 (based on Allred-Rochow electronegativity of M EN(M) and O EN(O) and equation (1) below:

EN(MOₓ)=(EN(M)^{0.5}+x EN(O)^{0.5})/(1/EN(M)^{0.5}+x/EN(O)^{0.5}) (1)

A preferred catalyst is KOH.

The basic catalyst component may be present in pure form, or may be supported on a carrier. Suitable carriers include C, SiO₂, Al₂O₃, ZrO₂, TiO₂, and other oxides and mixtures, or part of a compound (e.g. as mixed oxide).

A catalyst comprising K/ZrO₂, especially KOH/ZrO₂, is particularly preferred. More preferably, the KOH loading on the carrier may be greater than or equal to 0.5, 1, 3, 5 wt % and less than or equal to 30, 20, 15, 10, and preferably is from 5 to 15 wt %.

In the process of the invention, the C3-oxygenates are preferably diluted in water. Thus the use of an aqueous monopropylene glycol and aqueous 1,3-propanediol feed is advantageous, or water may be supplied separately to the reaction along with the C3-oxygenate feed. The C3-oxygenate feed may comprise a fraction, preferably an aqueous fraction, from a biomass conversion process.

Preferably the monopropylene glycol or 1,3-propanediol is diluted in water at a concentration of greater than 10, 20, 30, 40 or up to 50% v/v and at a concentration of less than 100, 90, 80, 70, 60 or 50% v/v.

The dehydration step is preferably carried out a temperature of from 325° C. to 450° C., more preferably from 350° C. to 420° C., and most preferably from 380° C. to 410° C.

The dehydration step may take place in a dehydration reactor and the reaction products therefrom may subsequently be fed to a separation unit, e.g. a distillation column, for extraction.

Allyl alcohol produced in the dehydration step may be extracted as a top stream along with water and any other oxygenates such as propanal and propanol. Preferably, allyl alcohol and water are extracted together, for example as a middle fraction. Having respective boiling points of 97° C. and 100° C., and potentially forming an azeotropic mixture, it is both economically and technically advantageous to recover allyl alcohol and water together rather than separating allyl alcohol from water prior to undertaking any oxidation to produce acrylic acid.

1-propanol which may also be formed during dehydration has a boiling point of 98° C. and may also be extracted from the distillation column together with the allyl alcohol/water mixture. Thus a combined feed of allyl alcohol, water and 1-propanol derived from the dehydration process, such as obtained as a middle fraction therefrom, may be introduced into an oxidation reactor.

Other dehydration products having lower boiling points than the “close boilers” of allyl alcohol, water and 1-propanol, such as propanal (boiling point of 48° C.), may be separated by distillation as a top fraction.

Any unconverted glycol is preferably recycled. For example, unconverted glycol may be removed, along with the least volatile (or heavy) components such as 2-ethyl-4-methyl-1,3-dioxolane, the ketal product of MPG and propanal, as a bottom fraction from the distillation column and then recycled to the dehydration reactor.

By not isolating the allyl alcohol from its close boilers, the distillation resistance of the dehydration “effluent” may be drastically reduced. Distillation resistance provides a useful means for determining whether the work up of a product mixture by distillation is economically viable and the concept thereof is discussed in J. P-Lange, ChemSusChem 2017, 10, 245-252.

Distillation resistance Ω_{prod} is based on the mass ratio of each component i over the target product fᵢ/f_{prod} and on the temperature gap ΔTᵢ [° C.] between their atmospheric boiling point and that of the next heavier component as per equation (2) below. Non-condensable products and the bottom stream are omitted from this calculation as these products do not need to undergo evaporation+condensation.

Ω_{prod}[1/° C.]=100*Σ(fᵢ/f_{prod})/ΔTᵢ (2)

It may be seen that the product of the dehydration step includes three close-boilers, namely allyl alcohol (97° C.), 1-propanol (98° C.) and water (100° C.). Their small ΔTi makes distillative separation challenging, as confirmed by Ωprod˜130 (Table 1).

TABLE 1
Distillation resistance of dehydration effluent
with separation of close boilers.

| | | | | R |
|---|---|---|---|---|
| | Mw | Tb | amount | /t |
| Name | g/mole | C. | t/t prod. | All. Alc |
| propanal | 58 | 48 | 0.52 | 1.1 |
| allyl | 58 | 97 | 1.00 | 100.0 |
| alcohol | | | | |
| 1- | | | | |
| propanol | 60 | 98 | 0.36 | 18.0 |
| H₂O | 18 | 100 | 6.00 | 6.8 |
| MPG | 76 | 188 | 0.65 | 2.0 |
| HE | 134 | 220 | 1.17 | |
| (dimer) | | | | |
| Total | | | 7.88 | 127.9 |

By recovering these close boiler components as a mixture so that they may be fed unseparated to the oxidation step, then the distillation resistance falls to 9.4 (Table 2).

TABLE 2
Distillation resistance of dehydration effluent
without separation of close boilers.

| | | | | R |
|---|---|---|---|---|
| | Mw | Tb | amount | /t |
| Name | g/mole | C. | t/t prod. | All. Alc |
| propanal | 58 | 48 | 0.52 | 1.0 |
| H₂O/allyl/1- | 58 | 100 | 7.36 | 8.4 |
| PrOH | | | | |
| MPG + HE | 76 | 188 | 0.65 | |
| Total | | | 7.88 | 9.4 |

It will be understood that a significant factor in the quantum of distillation resistance when allyl alcohol is separated derives from the need to evaporate 6 t of water per t of allyl alcohol (based on 50% MPG in water). Further efficiencies in the process may be obtained by either reducing the water dilution of the feed or by reaching higher yield per pass (assumed here at 30 mol %) or both.

Allyl alcohol produced by dehydration as per the above process may then be fed, preferably together with the water, with air or oxygen into an oxidation reactor to be converted to acrylic acid.

The oxidation step may be carried out at a temperature of from 250° C. to 450° C., preferably from 300° C. to 400° C., more preferably from 310° C. to 380° C., and most preferably from 315° C. to 360° C.

Acrylic acid may be suitably recovered from the oxidation reactor effluent, preferably extracted by means of absorption or reactive condensation.

When compared to other acrylic acid production routes using renewable feedstocks, the present process has a relatively high route efficiency.

In the present process, acrylic acid can be made from C3-oxygenates obtained from a renewable feedstock. That is, the present process provides a commercially useful means for obtaining acrylic acid other than from propene that would normally originate from a non-renewable, fossil feedstock. While acrylic acid could also be made from propene produced from a renewable feedstock, for example using propene produced from a sugar source (which is a renewable feedstock), after which the propene is oxidized into acrylic acid using conventional technologies as already discussed above, such an alternative route is less viable in terms of mass efficiency, carbon efficiency and/or fossil CO₂ intensity (or fossil CO₂ footprint).

Advantageously, the allyl alcohol feed comprises a fraction extracted from the dehydration process as hereinbefore described, which fraction may also comprise water and optionally 1-propanol.

The oxidation process itself results in a product mixture comprising close boilers, similar to the dehydration process. In the oxidation process, the close boilers comprise residual allyl alcohol and water (97° C. and 100° C. respectively), and also acrylic acid and propanoic acid (both 141° C.). The product mixture may be separated in a distillation column and acrylic acid extracted therefrom.

In order to reduce distillation resistance and thereby improve efficiency of the oxidation process, close boilers are preferably extracted together rather than undergo separation. Thus, unreacted allyl alcohol and water may be extracted together for subsequent recycling to the dehydration reactor. The product mixture from the oxidation reaction may further include acetic acid and this is also preferably extracted with the water/allyl alcohol for recycling to the dehydration reactor.

The distillation resistance is further reduced by extracting crude acrylic acid (that is acrylic acid together with propanoic acid) from the distillation column. If desired, the crude acrylic acid may subsequently be purified by isolating from any propanoic acid, for example, by known crystallisation methods.

As may be understood from Tables 3 and 4 below, the distillation resistance of the oxidation effluent is effectively reduced by about 50% when allyl alcohol and water are not extracted individually, but extracted together. Since dehydration of C3-oxygenates preferably takes place in an aqueous environment, it is efficient to recycle the allyl alcohol/water together from the oxidation effluent back into the dehydration process.

TABLE 3
Distillation resistance of oxidation effluent without separation
of close boilers acrylic and propanoic acids.

| | Mw | Tb | amount | R |
|---|---|---|---|---|
| Name | g/mole | C. | t/t prod. | /t AcrA |
| CO₂ + gas | 44 | −78 | na | |
| acetaldehyde | 44 | 20.2 | 0.01 | 0.0 |
| propanal | 58 | 48 | 0.01 | 0.2 |
| acrolein | 56 | 53 | 0.01 | 0.0 |
| allyl alcohol | 58 | 97 | 0.03 | 1.0 |
| H₂O | 18 | 100 | 3.13 | 18.4 |
| acetic acid | 60 | 117 | 0.1 | 0.4 |
| acryl./prop. | | | | |
| acid | 72 | 141 | 1.01 | 2.1 |
| unknown | 116 | 188 | 0.09 | |
| Total | | | 4.39 | 22.2 |

TABLE 4
Distillation resistance of oxidation effluent without
separation of acrylic and propanoic acids, and without
separation of acetic acid from water.

| | | Tb | amount | R |
|---|---|---|---|---|
| Name | g/mole | C. | t/t prod. | /t AcrA |
| CO₂ + gas | 44 | −78 | na | |
| acetaldehyde | 44 | 20.2 | 0.01 | 0.0 |
| propanal | 58 | 48 | 0.01 | 0.2 |
| acrolein | 56 | 53 | 0.01 | 0.0 |
| H₂O/allyl | 18 | 100 | 3.26 | 7.9 |
| alcohol | | | | |
| acryl./prop. | 72 | 141 | 1.01 | 2.1 |
| acid | | | | |
| unknown | 116 | 188 | 0.09 | |
| Total | | | 4.39 | 10.3 |

### EXAMPLES

### Example 1

Aqueous monopropylene glycol (50% in water) was dehydrated in the presence of a catalyst.

The catalyst was made from commercial monoclinic ZrO₂ (BET of 84 m²/g) obtained from Gimex Technische keramiek b.v. and KOH grains obtained from Sigma Aldrich. The catalysts with different weight percentages (0.1-10 wt %) of KOH were made by impregnation method. The required amount of KOH granules were diluted in approximately 20 mL of water and stirred by magnetic stirrer at a frequency of 450-500 rpm.

After dissolving, the required amount of ZrO₂ is added and stirring continued for 4-6 hours with the same stirring speed as mentioned above. For every catalyst solution, after 2 hours stirring, the temperature was raise to 100° C. to ease water evaporation. The resulting wet paste was dried in a vacuum oven overnight at 100° C. The catalysts were designated as, for example, 10KZrO₂ in which the numeral indicates the weight percentage of KOH on ZrO₂.

The catalytic tests were performed on a laboratory scale by using fixed-bed down flow quartz reactor (400 mm long, 4 mm id. and 6 mm od,) suspended in an electrical furnace. The catalyst with a particle size between 0.425-0.6 mm, mixed with silica beads of similar amount and size, is placed in the reactor, sandwiched between quartz wool. The liquid feed, monopropylene glycol (MPG, obtained from Sigma Aldrich) with required flow rates were pumped into the preheater maintained at 225° C. (above the boiling point of MPG, 188° C.), along with the carrier gas, preferably Ar or N₂, with a certain flow rate, before sending onto the catalyst bed.

The products were condensed using a cold trap placed at the bottom of the reactor and cooled to 10° C. Uncondensed vapours and the gases are sent into the gas chromatograph that is connected online. The liquid products of the reaction were quantified using high pressure liquid chromatography and the gaseous products were quantified by gas chromatography. All possible products were calibrated before being quantified.

Results showed production of allyl alcohol with a 47% yield and a conversion of 78%.

### Example 2

Allyl alcohol was oxidised to acrylic acid using a MoWVOₓ mixed oxide catalyst. A general reaction scheme is provided below.

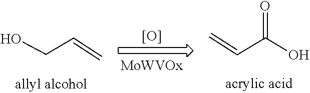

The catalyst was prepared as described in the literature [1]. Ammonium heptamolybdate (99%), and ammonium metatungstate (99%) were purchased from Alfa Aesar. Ammonium monovanadate was purchased from Merck. Typically, 2.6 g of ammonium monovanadate, 14.7 g of ammonium heptamolybdate and 2.7 g of ammonium metatungstate were dissolved in deionised water and then evaporated to dryness. This mixture was then calcined at 275° C. for 4 h and 325° C. for 4 h in air and N₂ respectively. Then the powder sample was crushed and sieved to make a particle size between 0.425-0.6 mm. The obtained catalyst is designated as MoWVOₓ. BET surface area is 12.5 m²/g based on XRD analysis.

The catalytic tests were performed on a laboratory scale by using a fixed-bed down flow quartz reactor (400 mm long, 4 mm id. and 6 mm od,) suspended in an electrical furnace. The catalyst was mixed with silica beads of similar amount and size, and placed in the reactor, sandwiched between quartz wool.

The liquid feed, aqueous solutions of allyl alcohol (AA, Sigma Aldrich), propionaldehyde (PAL, Sigma Aldrich), 1-propanol (POL, Merck) and monopropylene glycol (MPG, Sigma Aldrich) with required flow rates were pumped into the preheater maintained at 150° C. (in case of AA, PAL, POL) and 225° C. (for MPG) along with the carrier gas, preferably Ar or N₂, with a certain flow rate.

Pure oxygen is used as the oxidant and is connected to the feed stream after the stream is vaporised in the preheater and then the combination feed is allowed on to the catalyst bed. In a typical run, the reaction feed molar ratio of allyl alcohol:H₂O:argon:O₂ was 1:7.5:18.8:2.1.

The products were condensed using a cold trap placed at the bottom of the reactor and cooled to 5-10° C. Uncondensed vapours and the gases are sent into the gas chromatograph that is connected online. The liquid products of the reaction were quantified using high pressure liquid chromatography and the gaseous products were quantified by gas chromatography. All possible products were calibrated before being quantified.

The MoWVOₓ mixed oxide catalyst was tested for the oxidation of aqueous solution of allyl alcohol (30 vol %) at various reaction conditions, such as temperature and contact times.

Each reaction comprised two runs of 6-8 hours, each using a fresh catalyst: one from 340° C. to 280° C. and the other from 340° C. to 400° C. and back to 340° C.

Various process parameters were altered to determine their effect and to assist in optimising process conditions. The results are discussed in relation to the Figures, as follows:

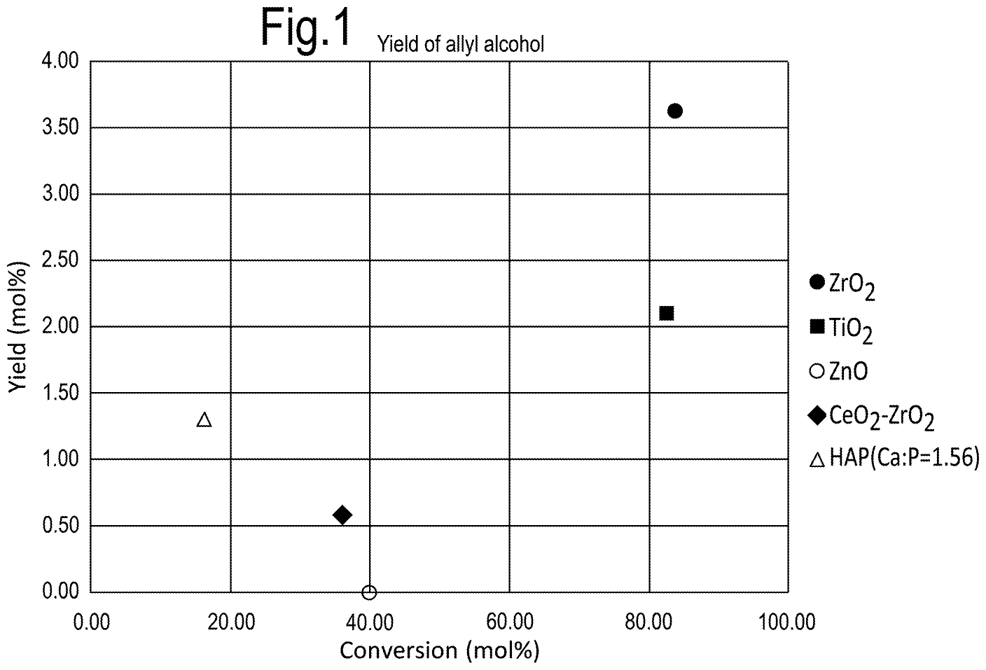

Referring to FIG. 1, the yield of allyl alcohol from monopropylene glycol using different catalysts is shown. It can be seen that a ZrO₂ catalyst provides the highest yield, as well as a high conversion. TiO₂ also provides a good conversion, but the yield was lower than for ZrO₂. The HAP (hydroxyapatite) catalyst is a hydrated mixed oxide of Ca(OH)₂ and H₂PO₄ with Ca/P atomic ratio of 1.56.

The results shown in FIG. 1 were derived from a reaction performed using a fixed bed reactor made out of glass and operated at atmospheric pressure at a temperature of 350° C. and a pressure of 1 atmosphere, the feed was pure monopropylene glycol fed at a rate of WHSV=2.35 h⁻¹ and carried with an argon flow at an Ar:MPG ratio of 8.45 (mol/mol).

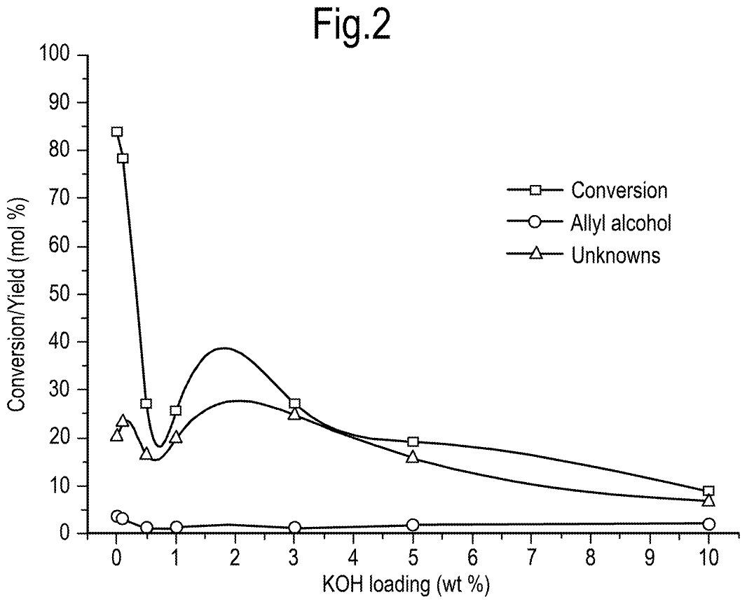

Referring to FIG. 2, it can be seen that increasing the loading of K on the ZrO₂ catalyst is beneficial as high KOH loadings increase selectivity to allyl alcohol by significantly decreasing formation of the ‘unknowns’ (i.e., heavy oligomeric materials that largely stay behind in the reactor and are quantified by poor closure of mass balance).

The results shown in FIG. 2 were derived from reactions performed at a temperature of 350° C. and a pressure of 1 atmosphere, the feed was pure monopropylene glycol, the carrier gas was argon (Ar:MPG 8.45 (mol/mol)) and WHSV was 2.35 g/g/h⁻¹. N₂ and CH₄ may alternatively be used as the carrier gas.

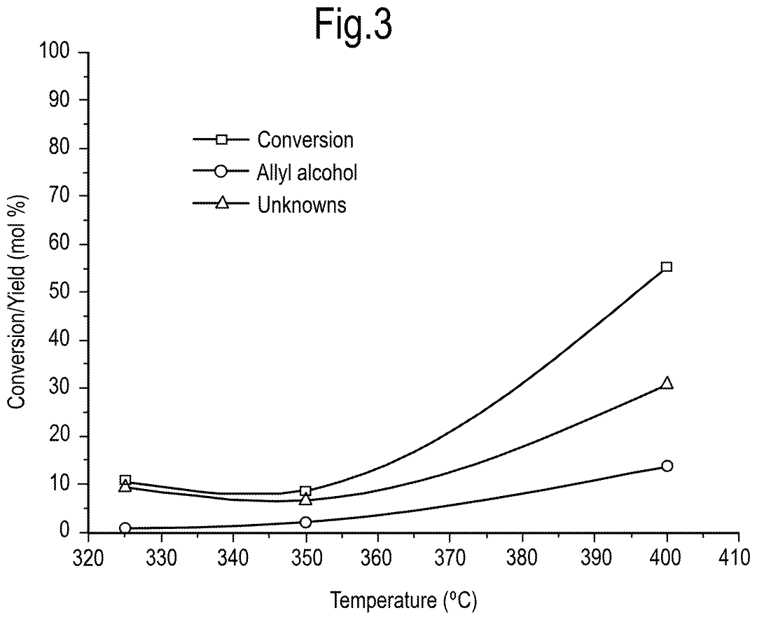

Referring to FIG. 3, it can be seen that an increase in temperature leads to an increase in the conversion and the yield of allyl alcohol. However, the amount of unknowns was also increased as temperature increased.

Accordingly, it will be appreciated that a balance of the reaction temperature and catalyst loading is required to optimise production of desired compounds, whilst avoiding increasing the formation of undesirable compounds.

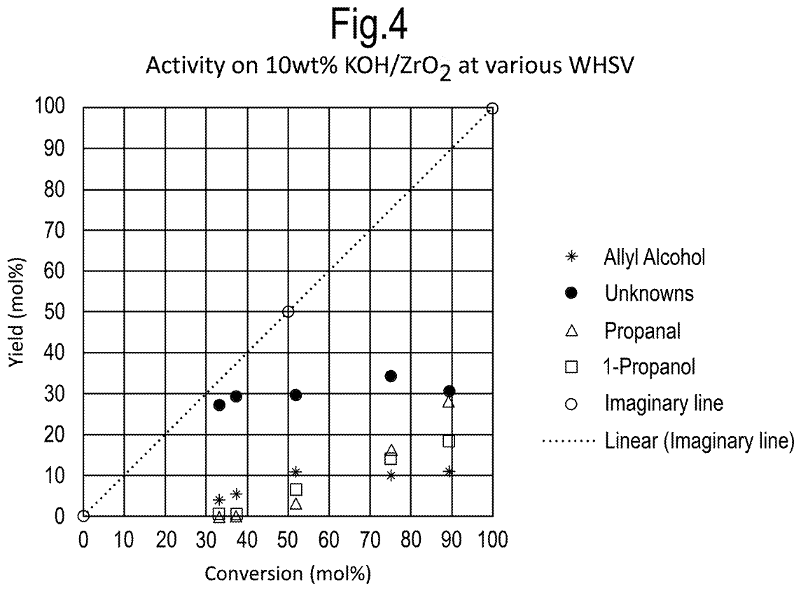

Referring to FIG. 4, it can be seen that WHSV has little appreciable effect on the yield of allyl alcohol after a particular value. The ‘unknowns’ observed at low conversion consists largely of dioxolanes, i.e. reversible adducts of propanal and unconverted MPG, which may revert to propanal/monopropylene glycol and subsequently to more propanal and allyl alcohol at higher conversion. The ‘unknowns’ reported at higher conversion consist of various condensation products. Yields of allyl alcohol of approximately 11% are obtained.

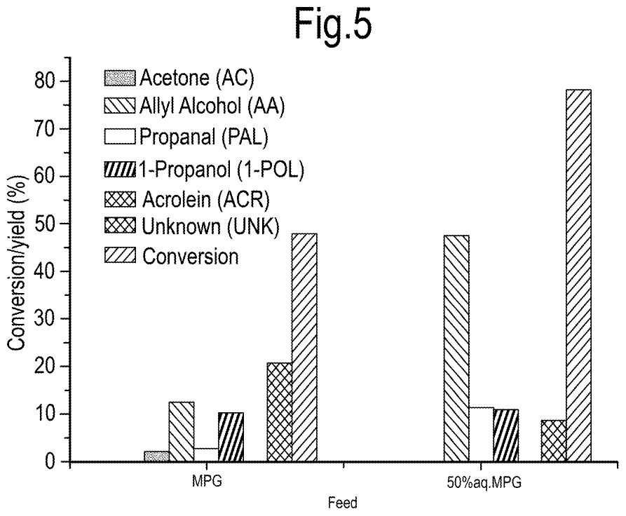

FIG. 5 shows that a monopropylene glycol feed diluted with water leads to a higher conversion of monopropylene glycol than an undiluted feed of monopropylene glycol, as well as a significantly higher yield of allyl alcohol (and propanal) and a lower yield of unknowns.

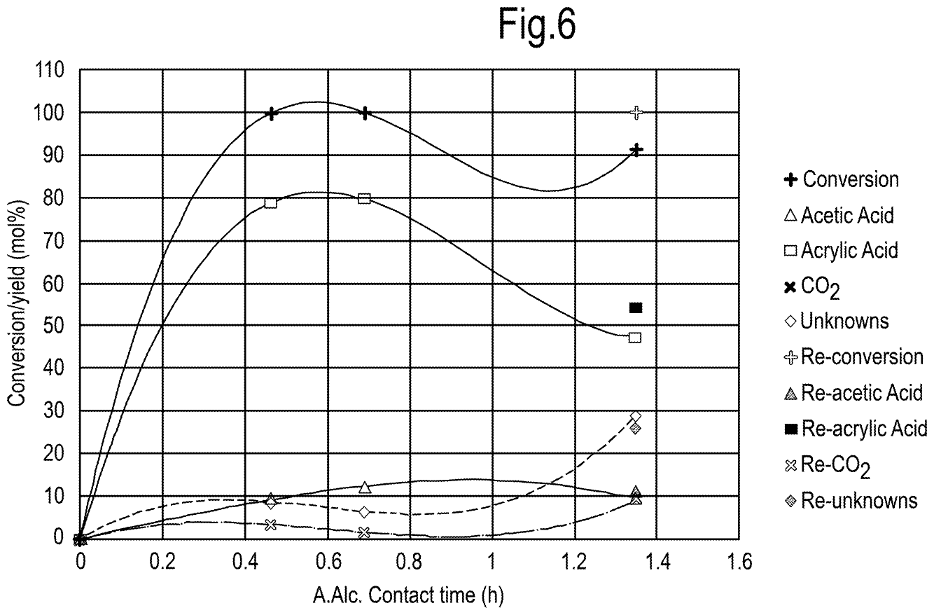

Referring to FIG. 6, the effect of temperature on allyl alcohol conversion on a MoWVOₓ catalyst is shown. In particular, it can be seen that a maximum yield of acrylic acid is observed at around 340° C.

By using optimised reaction conditions, the production of acrolein, proprionic acid, acetic acid, CO₂ and various ‘unknown’ other compounds is kept low. It is particularly beneficial to limit the production of such ‘unknown’ (often heavy) products, as they can lead to deactivation of the catalyst.

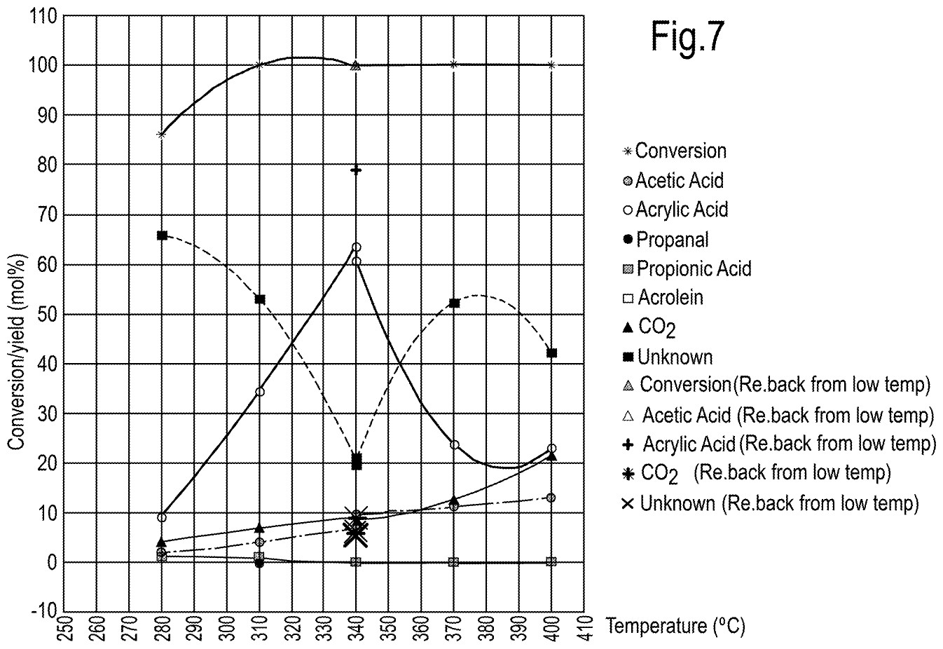

Referring to FIG. 7, the effect of contact time on allyl alcohol oxidation over a MoWVOₓ catalyst at 340° C. is shown.

It can be seen from FIG. 7 that higher contact times (i.e., above approximately 0.8 hours) lead to a decrease in the conversion of allyl alcohol to acrylic acid, for example, through an increase in the formation of unknown compounds instead. Accordingly, contact time is preferably maintained over a period of 0.4 to 0.8 hours.

Further comparisons are provided in Table 1 below, illustrating the effectiveness in terms of yield of using a highly basic catalyst, and the further benefits achieved when the dehydration step is carried out with such a highly basic catalyst in conjunction with using aqueous monopropylene glycol.

TABLE 1

| | | Yield (mol %) | | Conversion | |
|---|---|---|---|---|---|
| Catalyst | Conditions | Allyl Alc. | Propanal | (mol %) | Ref. |
| CeO₂ | 10 mol % aq MPG, 315° C., N₂ Carrier, | 0.22 | 2.42 | 22 | [1] |
| MgO | GHSV = 400 h⁻¹ | 0.2 | 2.7 | 10 | |
| ZrO₂ | | 1.3 | 3.25 | 65 | |
| Nb₂O₅ | | 0.8 | 11.4 | 20 | |
| Al₂O₃ | | 0.92 | 36.8 | 92 | |
| HZSM5 | | 1.65 | 41.8 | 55 | |
| Mordenite(3.3) | Pure MPG, 300° C., N₂ carrier, | 2.75 | 61.08 | 95 | [2] |
| | WHSV = 8.2 | | | | |
| Q10-SiW-30 (HPA) | Pure MPG, 200° C., N₂ carrier, W/F = | 0 | 66.42 | 98.4 | [3] |
| | 0.3/1.7 ghcm⁻³ | | | | |
| Commercial CeO₂ | Pure MPG(27 mmol/h), 325° C., N₂ | 1.4 | — | 3.2 | [4] |
| | carrier(73 mmol/h) | | | | |
| 10 wt % KOH/ZrO₂ | Pure MPG, 400° C., Ar Carrier, | 11 | 3.14 | 52 | Present |
| | WHSV = 2.35 h⁻¹ | | | | study |
| 10 wt % KOH/ZrO₂ | 50% aq MPG, 400° C., Ar Carrier, | 47 | 23 | 78 | Present |
| | Feed flow 0.495 g/h, 0.2 g catalyst, | | | | study |

### REFERENCES

[1]. Tao, L.-Z., et al., Comparison of gas-phase dehydration of propane polyols over solid acid-base catalysts. Catalysis Today, 2014. 234(0): p. 237-244.
[2]. Zhang, D., S. A. I. Barri, and D. Chadwick, Dehydration of 1,2-propanediol to propionaldehyde over zeolite catalysts. Applied Catalysis A: General, 2011. 400(1-2): p. 148-155.
[3]. Mori, K., Y. Yamada, and S. Sato, Catalytic dehydration of 1,2-propanediol into propanal. Applied Catalysis A: General, 2009. 366(2): p. 304-308.
[4]. Sato, S., et al., Selective dehydration of diols to allylic alcohols catalyzed by ceria. Catalysis Communications, 2003. 4(2): p. 77-81.

As can be seen from Table 1 above, the present inventors have been able to produce far higher yields of allyl alcohol from monopropylene glycol than in the prior art (references [1] to [4]). In this regard, the present inventors have been able successfully to shift the selectivity to allyl alcohol rather than other products, for example propanal.

In particular, use of a preferred KOH/ZrO₂ catalyst gives higher yields of allyl alcohol (11 mol %) compared to what is known in literature, and when diluted with water further improved yields (47 mol %) may be achieved.

It is believed that the presence of a highly basic catalyst, such as K catalyst, and water suppresses (i) transfer hydrogenation and (ii) formation of oligomers leading to the benefits hereinbefore described.

As shown in FIG. 7, a maximum yield of acrylic acid is observed at around 79%, which is far higher than previously reported yields.

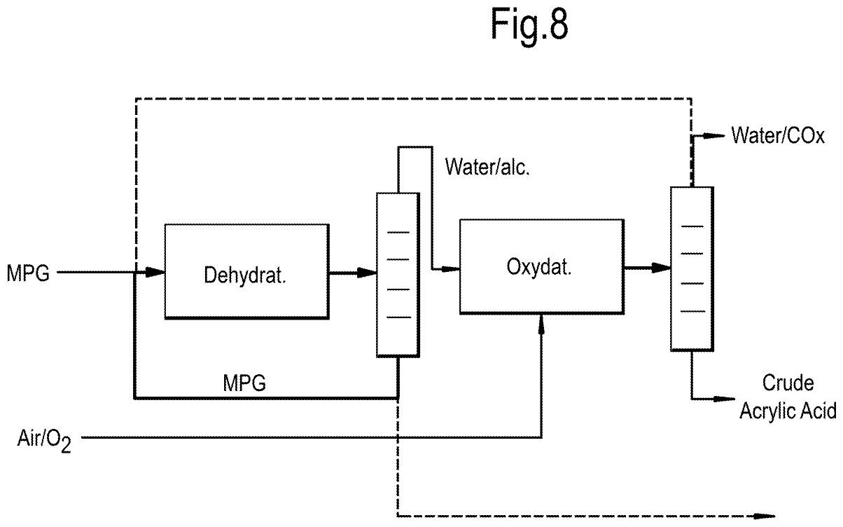

Referring to FIG. 8, a simplified process scheme showing the preparation of crude acrylic acid from aqueous monopropylene glycol is provided.

In the process scheme of FIG. 8, aqueous monopropylene glycol (1) is dehydrated (2); this dehydration (2) generally leads to the formation of allyl alcohol (4) and possibly also by-products, such as propanal and/or 1-propanol. The product(s) of the dehydration step (4) may be oxidised (5) in the presence of air/O₂ (8) to produce crude acrylic acid (7). Unconverted monopropylene glycol (3) is preferably removed (e.g., by condensation or distillation) such that it can be recycled and dehydrated again.

The dehydration step (2) is preferably performed at a moderate conversion per pass, so as to limit the formation of heavy by-products. Such by-products are preferably removed before the oxidation step.

The acrylic acid (7) is recovered preferably without condensing the water (6), for example using absorption or reactive condensation.

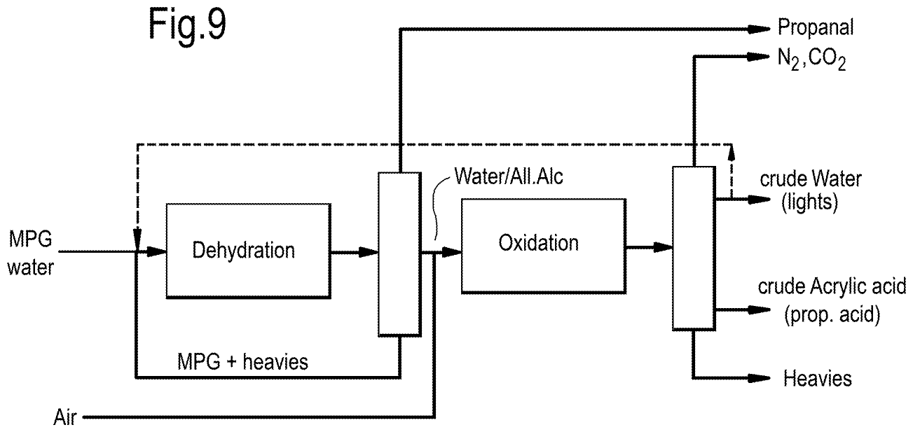

Referring to FIG. 9, an advanced process scheme showing the preparation of crude acrylic acid from aqueous monopropylene glycol is provided. It will be understood that the apparatus may comprise a series of separation units, such as a train of distillation columns, but for the purpose of illustration this is shown schematically as a single process feature.

In the process scheme of FIG. 9, aqueous monopropylene glycol is dehydrated leading to the formation of allyl alcohol and by-products, such as propanal and/or 1-propanol. Unreacted monopropylene glycol (MPG) is returned to the dehydration reactor along with other heavies. Propanal and other light components are extracted to be valorised e.g. as co-product. The allyl alcohol produced by dehydration is extracted as a middle fraction with water and fed to the oxidation reactor without separating from the water. Any 1-propanol produced as a by-product in the dehydration reaction is also transferred to the oxidation reaction in the allyl alcohol/water mixture without the need for separation. Air (or oxygen) is fed to the oxidation reactor to enable oxidation of the allyl alcohol to acrylic acid. The oxidation effluent is transferred to a further separation train from where gases such as nitrogen and carbon dioxide are vented off and the heavies or bottom fraction recycled back to the dehydration reactor. The oxidation effluent includes crude water which is separated as a light fraction and recycled to the dehydration reactor. The crude water potentially contains acetic acid but it is not required to remove the acetic acid before recycling. The oxidation effluent also contains the desired acrylic acid which is extracted without purification as a middle fraction. The extracted crude acrylic acid typically includes some propanoic acid and optionally this can be separated in a conventional process, such as by crystallisation, to obtain pure acrylic acid.

1. A process for producing allyl alcohol, the process comprising:
dehydrating a C3-oxygenate comprising monopropylene glycol or 1,3-propanediol;
wherein the dehydration is performed in the presence of a basic catalyst.

2. The process of claim 1, wherein the C3-oxygenate is diluted with water, preferably at a concentration of greater than 10, 20, 30, 40 or 50% and at a concentration of less than 100, 90, 80, 70, 60 or 50%.

3. The process of claim 1, wherein the basic catalyst comprises an element with an electronegativity of less than 2.0, more preferably less than 1.5 and most preferably less than 1.0, according to the Allred-Rochow scale of electronegativity.

4. The process of claim 3, wherein the element is from Group 1 and/or Group 2, preferably in the form of a metal oxide, hydroxide or mixture thereof, more preferably KOH.

5. The process of claim 3, wherein the basic element is carried on a support selected from the group consisting of SiO₂, Al₂O₃, ZrO₂, TiO₂, ZnO and CeO₂—ZrO₂ and mixture thereof, or on a carbon support.

6. The process of claim 5, wherein the catalyst is KOH/ZrO₂.

7. The process of claim 1, wherein the dehydration is conducted at a WHSV of ≥0.1 g/g/h and ≤10 g/g/h, preferably ≥0.3 g/g/h, and more preferably ≥1 g/g/h.

8. The process of claim 1, wherein the dehydrated C3-oxygenate comprising allyl alcohol is converted by oxidation into acrylic acid, preferably in an oxidation reactor.

9. The process of claim 8, wherein the dehydrated C3-oxygenate is obtained as a fraction of dehydration effluent, which fraction also comprises water, and said fraction is fed to an oxidation reactor for converting the dehydrated C3-oxygenate into acrylic acid.

10. The process of claim 8, wherein oxidation to acrylic acid is performed in the presence of a mixed oxide containing Mo, W, V or a mixture thereof as catalyst.

11. The process of claim 8, wherein acrylic acid is obtained as a fraction of oxidation effluent comprising crude acrylic acid.

12. The process of claim 8, wherein a further fraction of oxidation effluent comprising crude water is recycled to a dehydration reactor for dehydrating C3-oxygenates.

13. A biomass conversion process, the process comprising converting a biomass to produce a fuel product and monopropylene glycol and/or 1,3-propanediol as by-products, wherein the by-products are separated from the fuel product and converted by dehydration to allyl alcohol in the presence of a basic catalyst, and optionally oxidizing the allyl alcohol to acrylic acid.

14. The process of claim 12, wherein the dehydration is performed in aqueous conditions and allyl alcohol is not separated from water prior to oxidation to acrylic acid.

15. The process of claim 13, wherein the dehydration is performed in aqueous conditions and allyl alcohol and water are not separated from 1-propanol prior to oxidation to acrylic acid.
